(19) United States
(12) Patent Application Publication
(10) Pub. No.: US 2017/0238571 A1
(43) Pub. Date: Aug. 24, 2017

(54) A SYNERGISTIC COMPOSITION COMPRISING A MIXTURE OF BACTERIA OF THE GENERA LACTOBACILLUS AND PROPIONOBACTERIUM PARTICULARLY USEFUL TO REDUCE OR ELIMINATE PATHOGEN CONTAMINATION IN SOYBEN MEAL AND ITS DERIVATIVES; METHODS AND USES THEREOF

(71) Applicants: Molinos Río de la Plata S.A., Ciudad Autónoma de Buenos Aires (AR); Molinos IP S.A., Case Postale 29 Paudex (CH)

(72) Inventors: Francisco Colombatti, Santa Fe (AR); Luis Eduardo Palacios, Caba (AR); Ernesto Oscar Ventrici, Caba (AR)

(73) Assignees: Molinos Rio de la Plata S.A., Ciudad Autonoma de Buenos Aires (AR); Molinos IP S.A., Case Postale 29 Paudex (CH)

(21) Appl. No.: 15/306,031

(22) PCT Filed: Apr. 24, 2014
(86) PCT No.: PCT/EP2014/058344

§ 371 Date: Oct. 21, 2016

(51) Int. Cl. A23B 7/155; C12N 1/20; A23K 30/18; A01N 63/00; A23L 3/3571; A23K 10/18

(52) U.S. Cl. CPC A23B 7/155; A23L 3/3571; A23K 10/18; A23K 30/18; A01N 63/00; C12N 1/20; A23V 2002/00

(57) ABSTRACT

The invention discloses a synergistic composition comprising a mix of bacteria of the genera Lactobacillus and Propionibacterium which is particularly useful to reduce or eliminate contamination by bacteria of the genus Salmonella and fungi, thus also preventing the occurrence of mycotoxins in soybean meal and its derivatives.

### BACKGROUND OF THE INVENTION

1. Technical Field

This invention relates to compositions and methods useful to reduce or eliminate pathogen contamination in soybean meal and its derivatives. More specifically, the invention relates to a composition comprising a mix of bacteria of the genus Lactobacillus and the genus Propionibacterium and to a method of application of said composition.

2. Description of The State of The Art

The global soybean meal market comprises a total of about 62 million tons. Argentina leads the soybean meal market by exporting about 30 million tons, i.e., almost 50% of the total amount. Then, in order of importance, Argentina is followed by Brazil and the United States of America, with 22% and 15% respectively of the world market. These data not only represent the volume of this market, but also that 85% of the business is concentrated in 3 countries: Argentina, Brazil, and the U.S.

Currently soybean meal is considered a “feed ingredient” and the most important microbiological parameter assessed in this meal product is the presence of Salmonella. Mycotoxins are the second indicator of quality assessed with microbiological parameters. Keeping these parameters within specification is essential to avoid not only undesirable fines but also to avoid possible rejection of shipments and the high costs of meal treatment and decontamination. In addition to tangible costs like those mentioned above, there are also intangible costs, which are the reflection of product quality in supplier reputation.

The European Union has in place a system known as Rapid Alert System for Food and Feed (RASFF) which centralizes claims related to contaminated goods (with Salmonella or mycotoxins) detected at European ports. Centralization of information allows easy identification of those suppliers whose products are frequently contaminated, and the suppliers involved consequently face numerous problems accompanied by high financial losses,

In an industrial meal production plant there may be many sources of contamination. Addressing the solution to such problem requires multiple approaches, resulting in multiple “points of attack”. Among the most important control elements we could mention:
1—Preventing entry or spread of pathogens in the facility.
2—Increasing astringency of hygiene practices,
3—Implementing designs allowing easy cleaning and disinfection of equipment and facilities.
4—Preventing or minimizing growth of pathogens in the facility.
5—Establishing a control program.
6—Validating control measures taken to eliminate pathogens.
7—Establishing procedures for the verification of different pathogen controls, as well as for any necessary corrective actions.

The presence of Salmonella in a meal product or in other low-water activity products is a concern because even small concentrations of Salmonella in food can cause deceases. Salmonella can persist for extended periods of time in low-moisture products, and undoubtedly, this dangerous ability of the pathogen makes it an etiologic agent that can be difficult to control, Similarly to Salmonella, mycotoxins are highly stable molecules, These dangerous metabolites are synthesized and excreted in the matrix by certain mycotoxin-producing fungi,

Contrary to what occurs with Salmonella, the presence of mycotoxins in meal products does not imply the actual presence of a fungus, although it can be argued that at some point it was present in the matrix. However, the stability of mycotoxins turns these molecules into agents that are as hazardous as Salmonella.

The use of Lactobacillus in the agricultural and food industries has been previously described. The use of Lactobacillus has also been described as inhibiting the growth of Salmonella in machinery and manufacturing processes of food products. The prior art indicates that the effectiveness of using Lactobacillus as a Salmonella inhibitor depends on both the product to be treated and the environmental conditions thereof.

Commercial products used for treating Salmonella are comprised of mixes of short-chain organic acids. Propionic acid has shown to have a strong effect on Salmonella. Propionibacterium freudenreichii has the ability to produce significant amounts of propionic acid (yields of up to 80 g/L have been reported in strains not subjected to mutagenesis). However, changing the complex metabolism of this microorganism to produce propionic acid, involves changes in technical variables that must be performed at specific stages of cell growth. In addition, P. shermanii produces metabolites other than propionic acid, which prevent the development of other microorganisms.

In particular, Argentine Patent No AR061534B1 of 07/19/2012 discloses a composition useful to eliminate Salmonella comprising:
Lactobacillus casei ATCC 393
Lactobacillus fermentum ATCC 9338
Lactobacillus gasseri ATCC 33323
Lactobacillus plantarum ATCC 14917
Lactobacillus rhamnosus ATCC 7469

It is then necessary in the market to count with new products and improved methods to minimize or prevent the occurrence of pathogens in soybean meal and its derivatives. Such products should be easily formulated and applied, should have a broad spectrum, be harmless to human health, should have a residual effect, and above all, they should be inexpensive.

Given that the only microbiological parameters regulated nowadays in the meal products market are related to the presence of Salmonella and mycotoxins produced by fungi, new product development should be focused mainly on these agents,

### SUMMARY OF THE INVENTION

The object of this invention is a synergistic composition comprising a mix of bacteria of the genera Lactobacillus and Propionibacterium which is particularly useful to eliminate bacterial contamination by Salmonella and fungi in soybean meal products and derivatives thereof, said composition comprising:
Lactobacillus casei ATCC 393
Lactobacillus fermentum ATCC 9338
Lactobacillus gasseri ATCC 33323
Lactobacillus plantarum ATCC 14917
Lactobacillus rhamnosus ATCC 7469
Propionibaclerium freudenreichii subsp. shermanii ATCC 9614

The composition of the invention has an excellent performance regarding the issues set forth above from paragraph 4 to 7. The Lactic-Propionic mix described herein can be used to prevent the growth of Salmonella in a production plant, and it is easily applied by fumigation. Establishing a suitable plant-fumigation program complements the invention.

It is important to remark that the strains used in the invention are classified as GRAS (Generally Recognized as Safe) by the FDA, which evidences the safety of the composition, which can be easily handled without health risks.

### BRIEF DESCRIPTION OF DRAWINGS

### Abbreviations:

PCR=Polymerase Chain Reaction; CFU=Colony Forming Units; BPW=Buffered Peptone Water; SS Agar=Salmonella-Shigella Agar; DBM=Moisture Content on Dry Basis; MRS=de Man, Rogosa and Sharpe; ND=Not detected/detectable.

Values shown in Figures are means of three independent determinations. Standard deviations were in all cases less than 15% of the respective mean values.

FIG. 1, Results of PCR tests of different genomic samples extracted upon completion of fermentations, Reactions were run on a 1.5% agarose gel, Ethidium bromide was used as a fluorophore. Each band of the ladder has registered on them the sizes of the base-pair fragments.

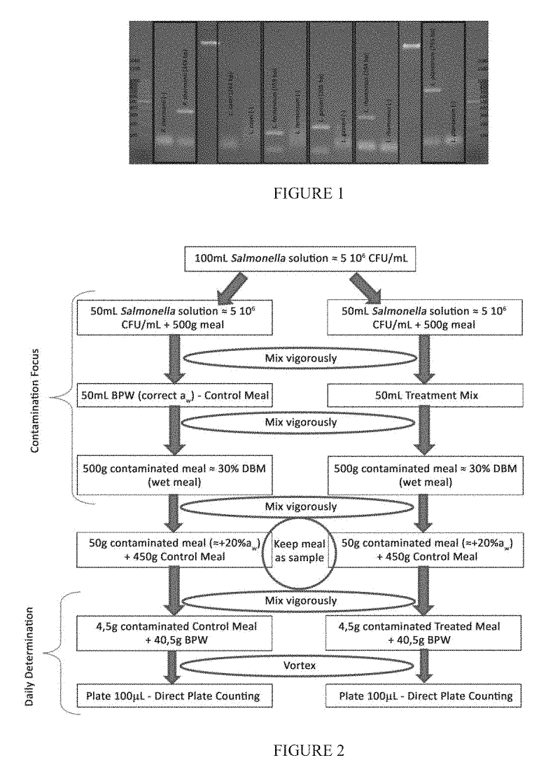

FIG. 2. Protocol used for contamination and subsequent detection of microorganisms in soybean meal.

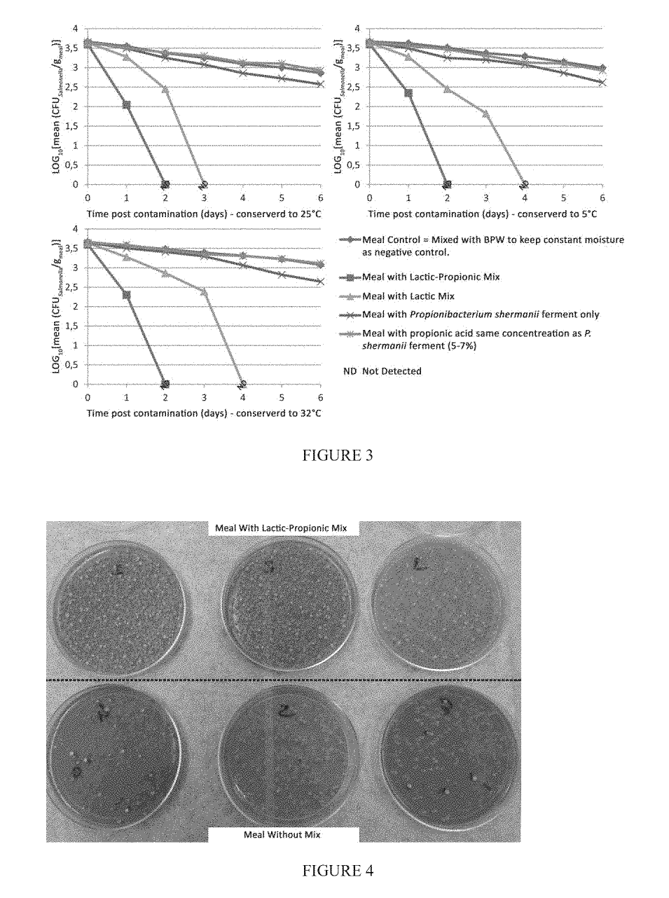

FIG. 3. Curves obtained by quantifying the concentration of Salmonella in soybean meal with 12% DBM, after having applied the protocol described in FIG. 2.

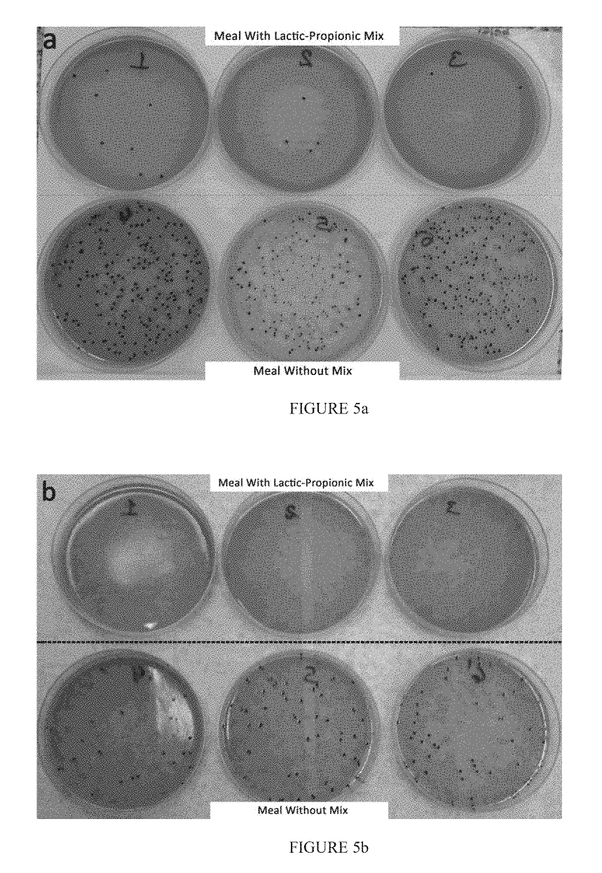

FIG. 4 shows the results obtained after inoculating 100μL obtained upon completion of the protocol shown in FIG. 2, on MRS agar. The colonies belong to the genus Lactobacillus and the genus Propionibacterium.

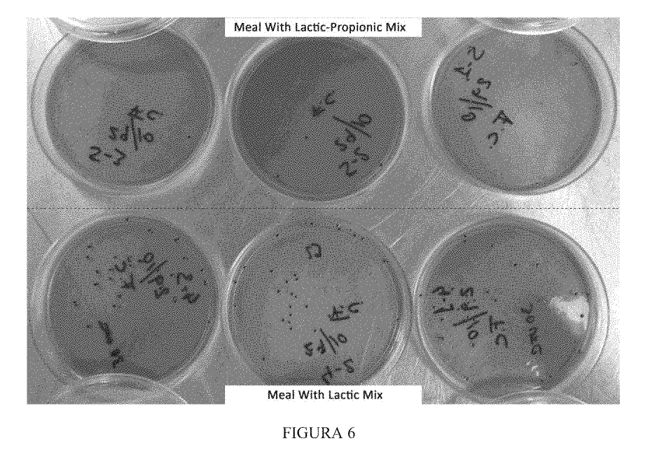

FIG. 5. Result of the plates obtained after performing the protocol described above in soybean meal. a: Comparison of Lactic-Propionic mix with control conditions 24 h after contamination. b: Comparison of Lactic-Propionic mix after 48 h of contamination.

FIG. 6, Comparison of Salmonella concentration 24 hours after completion of the contamination protocol (FIG. 2) with different mixes (Lactic-Propionic and Lactic mixes)

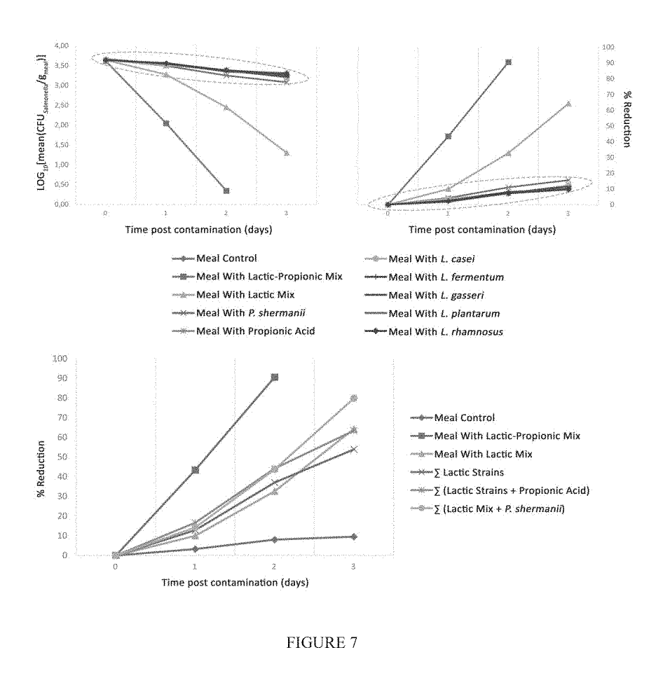

FIG. 7 shows a comparison of the effects sought by the invention. Top: Reduction of Salmonella caused by Lactic-Propionic mixes, by Lactic mix, by ferments obtained separately and by Propionic acid respectively. Evolution of Salmonella content as a reduction percentage of initial CFUs, Bottom: Synergistic effect, The effect of the mixtures was higher than the added effects of the individual components,

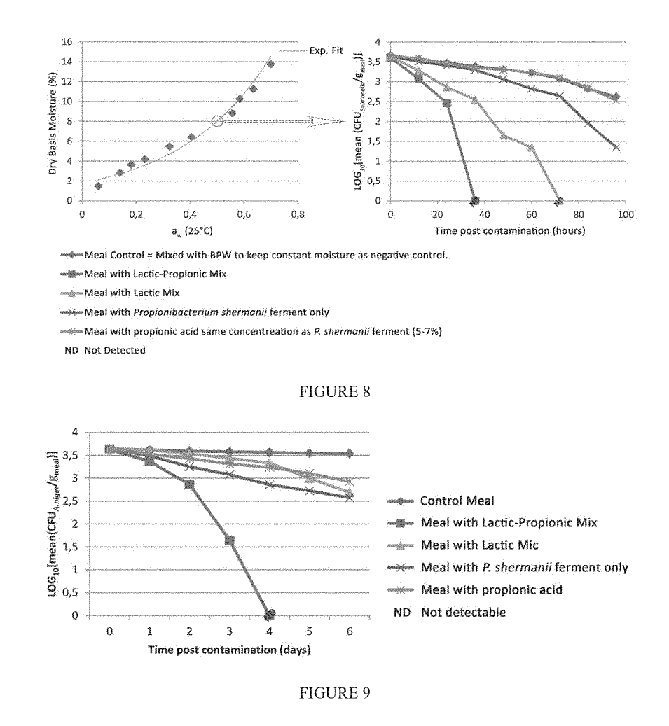

FIG. 8. Relationship between DBM and aw at 25° C. The circle shows breaking point of the curve, at 8% DBM. The arrow indicates the result obtained after drying soybean meal up to 8% DBM and quantifying the evolution of Salmonella in such matrix at 25° C.

FIG 9. Curves obtained upon determination of A. niger in premixed meals with various protective solutions.

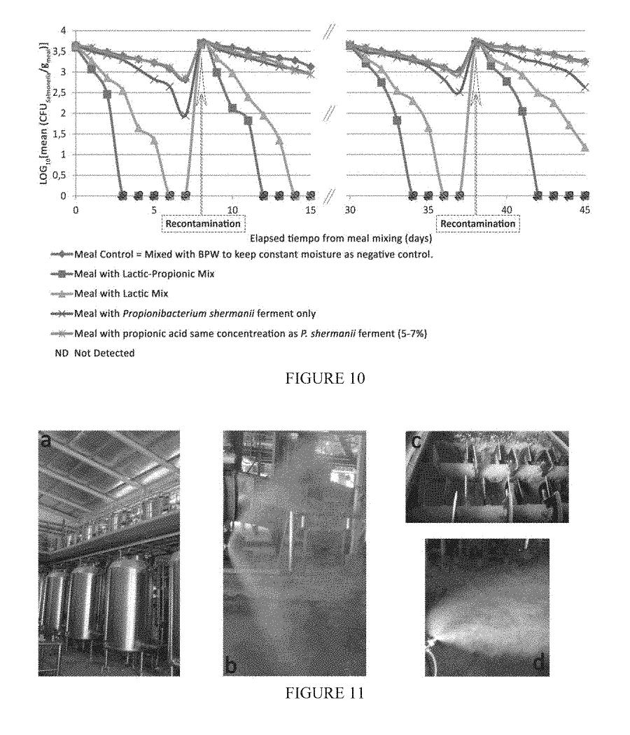

FIG. 10. Residual effect. Curves obtained upon determination of Salmonella in soybean meal pretreated with various solutions.

FIG. 11 shows: a—Fermentation Plant, with the six fermenters. b—Side-view of ferment cloud produced by pneumatic nozzles, freshly dried meal breaks through the cloud. c—Screw mixer where soaked meal is mixed with the Lactic-Propionic solution. d—Top view of a nozzle during application.

### DETAILED DESCRIPTION OF THE INVENTION

### Abbreviations:

PCR=Polymerase Chain Reaction; CFU=Colony Forming Units; BPW=Buffered Peptone Water; SS Agar=Salmonella-Shigella Agar; DBM=Dry Basis Moisture Content; MRS=de Man, Rogosa and Sharpe; ND=Not detected/detectable.
MRS-agar composition: Proteose Peptone 10 g/L, Meat Extract 8 g/L, Yeast Extract 4 g/L, Glucose 20 g/L, Sorbitan Monoleate 1 mL/L, K₂HPO₄ 2 g/L, Sodium Acetate 5 g/L, Ammonium Citrate 2 g/L, MgSO₄ 0.2 g/L, MnSO_{4 0.05} g/L, Agar 13 g/L.
Modified MRS for fermentation composition: (NH₄)NO₃ 1 g/L, Yeast Extract 20 g/L, Glucose 30 g/L, Sorbitan Monoleate 1 mL/L, K₂HPO₄ 2 g/L, Sodium Acetate 5 g/L, MgSO₄ 0.2 g/L, MnSO₄ 0.05 g/L.
Salmonella-Shigella Agar composition: Pluripeptone 5 g/L, Meat Extract 5 g/L, Lactose 10 g/L, Bile Salts Mixture 8.5 g/L, Sodium Citrate 8.5 g/L, Na₂S₂O₃ 8.5 g/L, Ferric Citrate 1 g/L, Brilliant Green 0.00033 g/L, Neutral Red 0.025 g/L, Agar 13.5 g/L.
Czapek-Dox Agar Composition: Saccharose 30 g/L; NaNO₃ 3 g/L, K₂HPO₄ 1 g/L, MgSO₄ 0.5 g/L, MgCl₂ 0.5 g/L, FeSO_{4 0.01} g/L, Agar 15 g/L.

### Characterization of Strains Used in This Invention

The following strains were used:
Lactobacillus casei ATCC 393
Lactobacillus fermentum ATCC 9338
Lactobacillus gasseri ATCC 33323
Lactobacillus plantarum ATCC 14917
Lactobacillus rhamnosus ATCC 7469
Propionibacterium freudenreichii subsp. shermanii ATCC 9614
Lactic-Propionic mix: L. easel, L. fermentum, L. gasseri, L. plantarum, L. rhamnosus, P. shermanii. Equal amounts of each ferment obtained at 36 h and 96 h respectively.
Lactic mix: L. casei, L. fermentum, L. gasseri, L. plantarum, L. rhamnosus, Equal amounts of each ferment obtained at 36 h,
P. shermanii ferment: Product obtained after 96 h of fermentation of P. shermanii strain in two stages; an anaerobic stage, and an aerobic stage with low oxygen concentrations.
Propionic Add: Solution used as P. shermanii fermentation blank (5-7%).

The validity ranges of the synergistic composition are from a 10⁵ to a 10¹¹ concentration with the clear implication that the higher cell concentration, the greater the effectiveness of the product obtained. In turn, the composition described herein comprises equal amounts of ferments reaching similar concentrations in CFU/mL; however, we have demonstrated that changing the ratios the product also works. As in the case of the concentration, as we move away from the ratios described herein, the product becomes less effective.

In a mix of different strains whose total cell concentration is in the range of 10⁵-10¹¹ CFU/m L, the most concentrated strain should not be more than 1000 times more concentrated (in CFU/mL) than the least fermented strain,

Specific oligonucleotides were designed to check that each ferment effectively belonged to each tested strain and in order to avoid cross contamination. All fermentations were completed simultaneously, and, in the case of P. shermanii, fermentation took 96 h, whereas in the case of lactic acid fermentations lasted 36 h.

PCR identification was performed using specific oligonucleotides to amplify the 16S DNA region in the case of lactic bacteria, and in the 16S-23S intergenic region in the case of P. shermanii. Genomic DNA extraction was performed using a protocol involving the use of mutanolysin,

The following table contains the oligonucleotides used in the invention:

| | | | Product | Target |
|---|---|---|---|---|
| | | Sequence (5′→3′) | Lenght (bp) | region |
| L. casei | Forward primer | CGAGTTTTGGTCGATGAACGGTGC | 242 | 16S |
| | Reverse primer | ATCATCGCCTTGGTGAGCCGT | | |
| L. fermentum | Forward primer | CAACGAGTGGCGGACGGGTG | 159 | 16S |
| | Reverse primer | TGCACCGCAGGTCCATCCAG | | |
| L. gasseri | Forward primer | GGCGGCTCTCTGGTCTGCAA | 205 | 16S |
| | Reverse primer | CGGGCCCCCGTCAATTCCTT | | |
| L. plantarum | Forward primer | GGACCGCATGGTCCGAGCTT | 755 | 16S |
| | Reverse primer | CGGGCCCCCGTCAATTCCTT | | |
| L. rhamnosus | Forward primer | TGGACCCGCGGCGTATTAGC | 294 | 16S |
| | Reverse primer | TGCCTACGTATTACCGGCTGGC | | |
| P. shennanii | Forward primer | TGACCGTAGATTGTCGGCTG | 182 | Intergenic |
| | Reverse primer | CAAACACGGGGAACAACCAC | | 16S-23S |

### EXAMPLE

### Effectiveness of The Mix of The Invention Against Different Strains of Salmonella in Soybean Meal (FIG. 2)

In order to assess the effectiveness of the mix of the invention in the matrix of interest, an appropriate working protocol was prepared. Initially, 500 g of soybean meal were infected with 50 mL of Salmonella solution whose composition was: 10⁶ CFU_{S. typhimurium}/mL, 10⁶ CFU_{S. enteritidis} /mLand 10⁶ CFU_{S. heidelberg} /mL in equal amounts. After mixing the Salmonella solution with the meal, 50 mL of different solutions were added, and in the control treatment only Buffered Peptone Water was added to have the same moisture content in all the samples. 50 g of the wet meal obtained (≈25% moisture content on dry basis) were mixed within the dry meal (≈10% moisture content dry basis) and were stirred for 10 minutes. Thus, not only a similar contamination to that occurring in the plant (through sources of infection) was ensured, but also a final meal product with similar moisture to that of the meal just coming out of the dryer was obtained. Daily determinations of Salmonella colony forming units were performed on this contaminated meal. To this end, 40.5 g of BPW to 4.5 g of the resulting meal were added and vigorously stirred, and then 100μL of this solution, were plated onto Salmonella-Shigella agar (SS Agar). When microorganisms were used in the protecting mixes, concentrations were carefully balanced, and the amount of bacteria added was always the same.

Simultaneously, bacteria were counted and tracked in a MRS (Man, Rogosa and Sharpe) medium. This culture medium allows the growth of lactic acid bacteria and propionic bacteria. In this way, tolerance of the three bacterial mixes tested was assessed.

Under extreme conditions (quantified as temperature) the Lactic-Propionic mix has an advantage against Salmonella. When the meal infected with Salmonella was subjected to extreme temperatures as low as 5° C. or as high as 32° C., the propionic-lactic mix showed a better performance than the lactic mix. In addition, it is clear that Salmonella has also a different behavior at extreme temperatures, under stringent moisture conditions. Such behavior is complex, and clearly responds to the different structures that this microorganism may adopt.

Protective solutions, as understood herein, are the mixes above described as: Lactic-Propionic mix, Lactic mix, BPW (as control), Propionic ferment, and 5-7% propionic acid depending on the concentration obtained during propionic fermentation (propionic fermentation blank). In the cases in which solutions with ferments were used for protection purposes, cell concentration was of about 10⁸ CFU/mL. For example, to make up 50 mL of the Lactic-Propionic mix, 8.33 mL of the ferment obtained from each strain, with values of about 10⁸ CFU/mL, were mixed together. To make up 50 mL of the lactic mix, 10 mL of the ferment obtained from each strain, with concentration values of about 10⁸ CFU/mL were mixed together.

Synergistic Effect: Independent Ferments vs. Mixes (FIG. 7)

Using the protocol described above, the effects caused by the individual strains were assessed and then compared with the effect obtained after mixing them together. The above protocol does not allow to detect less than 100 CFU/g, since it has 2 dilutions of 1/10 (4.5 g in 40.5 g of BPW buffer, and then 100 μL are taken and finally brought to 1 mL). In order to come closer to the actual values, whenever a ND value was obtained using the methodology described above, a new dilution factor was applied; each time by adding only 18 g of buffer (stirring thoroughly and then spinning), and 200 μL of this solution were plated for recounting. Thus, sensitivity was increased tenfold. This new protocol was only used to determine the effect of the different mixes. In turn, the results obtained herein are shown using the following formula:

$$\{ \frac{[ {log}_{10} ( mean CFU ) {t}_{0} ] - [ {log}_{10} ( mean CFU ) t ]}{[ {log}_{10} ( mean CFU ) {t}_{0} ]} \} * 100$$

Thus, the percentage of Salmonella elimination in meal was calculated for different mixes, as well as for each ferment individually. The following TABLE is related to FIG. 7.

TABLE 1
25° C. - 12% DBM

| | Meal Control | | |
|---|---|---|---|
| Time | Average | LOG10 | Reduction |
| (days) | (n = 3) | (CFU/g) | % |
| 0 | 208.33 | 3.67 | 0.00 |
| 1 | 158.67 | 3.55 | 3.22 |
| 2 | 106 | 3.37 | 8.01 |
| 3 | 80.33 | 3.25 | 11.27 |
| 4 | 58 | 3.09 | 15.57 |
| 5 | 45.33 | 3.00 | 18.05 |
| 6 | 32.67 | 2.86 | 21.94 |

25° C. - 12% DBM

| | Meal With Lactic-Propionic Mix | | |
|---|---|---|---|
| Time | Average | LOG10 | reduction |
| (days) | (n = 3) | (CFU/g) | % |
| 0 | 180.33 | 3.60 | 0.00 |
| 1 | 5 | 2.05 | 43.22 |
| 2 | *ND | 0.34 | 90.50 |
| 3 | *ND | — | <90.5 |
| 4 | *ND | — | <90.5 |
| 5 | *ND | — | <90.5 |
| 6 | *ND | — | <90.5 |

25° C. - 12% DBM

| | Meal With Lactic Mix | | |
|---|---|---|---|
| Time | Average | LOG₁₀ | reduction |
| (days) | (n = 3) | (CFU/g) | % |
| 0 | 196.67 | 3.64 | 0.00 |
| 1 | 85.33 | 3.28 | 9.98 |
| 2 | 12.67 | 2.45 | 32.59 |
| 3 | 0.9 | 1.30 | 64.26 |
| 4 | 0.4 | 0.95 | 73.94 |
| 5 | 0.2 | 0.65 | 82.21 |
| 6 | 0.14 | 0.49 | 86.46 |

25° C. - 12% DBM

| | Meal With P. shermanii | | |
|---|---|---|---|
| Time | Average | LOG₁₀ | reduction |
| (days) | (n = 3) | (CFU/g) | % |
| 0 | 200 | 3.65 | 0.00 |
| 1 | 140 | 3.49 | 4.25 |
| 2 | 80 | 3.25 | 10.91 |
| 3 | 54 | 3.08 | 15.59 |
| 4 | 32.43 | 2.86 | 21.66 |
| 5 | 23.87 | 2.72 | 25.31 |
| 6 | 16.85 | 2.57 | 29.45 |

25° C. - 12% DBM

| | Meal With Propionic Acid | | |
|---|---|---|---|
| Time | Average | LOG₁₀ | reduction |
| (days) | (n = 3) | (CFU/g) | % |
| 0 | 199.67 | 3.65 | 0.00 |
| 1 | 145 | 3.51 | 3.81 |
| 2 | 110 | 3.39 | 7.10 |
| 3 | 90 | 3.30 | 9.49 |
| 4 | 60.67 | 3.13 | 14.22 |
| 5 | 57 | 3.10 | 14.93 |
| 6 | 38.33 | 2.93 | 19.76 |

25° C. - 12% DBM

| | reduction % | | | | | |
|---|---|---|---|---|---|---|
| | | MW | | | | |
| | | Lactic- | MW | | Σ(Lactic + | Σ(Lactic |
| Time | Meal | Propionic | Lactic | ΣLactic | Propionic | Mix + P. |
| (days) | Control | Mix | Mix | Strains | Acid) | shermanii) |
| 0 | 0 | 0 | 0 | 0 | 0 | 0 |
| 1 | 3.22 | 43.40 | 9.98 | 12.82 | 16.64 | 14.23 |
| 2 | 8.01 | 90.53 | 32.59 | 37.03 | 44.13 | 43.69 |
| 3 | 9.50 | *ND | 64.26 | 53.90 | 63.40 | 79.85 |

25° C. - 12% DBM

| | Meal Control | | |
|---|---|---|---|
| Time | Average | LOG10 | reduction |
| (days) | (n = 3) | (CFU/g) | % |
| 0 | 208.33 | 3.67 | 0.00 |
| 1 | 158.67 | 3.55 | 3.23 |
| 2 | 106 | 3.37 | 8.01 |
| 3 | 93.33 | 3.32 | 9.51 |

25° C. - 12% DBM

| | Meal With Lactic-Propionic Mix | | |
|---|---|---|---|
| Time | Average | LOG10 | reduction |
| (days) | (n = 3) | (CFU/g) | % |
| 0 | 185.33 | 3.61 | 0.00 |
| 1 | 5 | 2.05 | 43.41 |
| 2 | 0.099 | 0.34 | 90.53 |
| 3 | 0.099 | 0.34 | 90.53 |

25° C. - 12% DBM

| | Meal With Lactic Mix | | |
|---|---|---|---|
| Time | Average | LOG10 | reduction |
| (days) | (n = 3) | (CFU/g) | % |
| 0 | 196.67 | 3.64 | 0.00 |
| 1 | 85.33 | 3.28 | 9.96 |
| 2 | 12.67 | 2.45 | 32.71 |
| 3 | 0.9 | 1.30 | 64.26 |

25° C. - 12% DBM

| | Meal With P. shermanii | | |
|---|---|---|---|
| Time | Average | LOG10 | reduction |
| (days) | (n = 3) | (CFU/g) | % |
| 0 | 201.33 | 3.65 | 0.00 |
| 1 | 140.67 | 3.49 | 4.27 |
| 2 | 80 | 3.25 | 10.98 |
| 3 | 54.33 | 3.08 | 15.58 |

25° C. - 12% DBM

| | Meal With Propionic Acid | | |
|---|---|---|---|
| Time | Average | LOG₁₀ | reduction |
| (days) | (n = 3) | (CFU/g) | % |
| 0 | 199.67 | 3.65 | 0.00 |
| 1 | 145 | 3.51 | 3.81 |
| 2 | 110 | 3.39 | 7.10 |
| 3 | 90 | 3.30 | 9.49 |
| | Meal With L. casei | | |
| Time | Average | LOG10 | reduction |
| (days) | (n = 3) | (CFU/g) | % |
| 0 | 199.76 | 3.65 | 0.00 |
| 1 | 145 | 3.51 | 3.81 |
| 2 | 110 | 3.39 | 7.10 |
| 3 | 70.67 | 3.20 | 12.37 |
| | Meal With L. fermentum | | |
| Time | Average | LOG10 | reduction |
| (days) | (n = 3) | (CFU/g) | % |
| 0 | 198.67 | 3.64 | 0.00 |
| 1 | 162.33 | 3.56 | 2.41 |
| 2 | 100.67 | 3.35 | 8.10 |
| 3 | 86.67 | 3.28 | 9.88 |
| | Meal With L. gasseri | | |
| Time | Average | LOG10 | reduction |
| (days) | (n = 3) | (CFU/g) | % |
| 0 | 196.67 | 3.64 | 0.00 |
| 1 | 158.67 | 3.55 | 2.56 |
| 2 | 105.33 | 3.37 | 7.45 |
| 3 | 74.67 | 3.22 | 11.55 |
| | Meal With L. plantarum | | |
| Time | Average | LOG10 | reduction |
| (days) | (n = 3) | (CFU/g) | % |
| 0 | 194.33 | 3.64 | 0.00 |
| 1 | 167.67 | 3.57 | 1.76 |
| 2 | 106 | 3.37 | 7.24 |
| 3 | 82 | 3.26 | 10.31 |
| | Meal With L. rhamnosus | | |
| Time | Average | LOG10 | reduction |
| (days) | (n = 3) | (CFU/g) | % |
| 0 | 197.67 | 3.64 | 0.00 |
| 1 | 163.33 | 3.56 | 2.28 |
| 2 | 108.67 | 3.38 | 7.13 |
| 3 | 87 | 3.29 | 9.78 |

### Effectiveness of Mixes at Different Temperatures (FIG. 3)

The following tests were carried out to assess the effectiveness of different mixes under different conditions. To assess extreme temperatures, meal products were stored at 5, 25, and 32° C., after contamination and protection, respectively. Samples were taken every 24 hours and triplicate determinations of Salmonella, lactic and propionic bacteria were made. Table 2 below shows the results illustrated in FIG. 3.

TABLE 2

| | | | MW Lactic- | | | | MW P. | | MW Propionic | |
|---|---|---|---|---|---|---|---|---|---|---|
| | Meal Control | | Propionic Mix | | MW Lactic Mix | | shermanii | | Acid | |
| Time | Average | LOG10 | Average | LOG10 | Average | LOG10 | Average | LOG10 | Average | LOG10 |
| (days) | (n = 3) | (CFU/g) | (n = 3) | (CFU/g) | (n = 3) | (CFU/g) | (n = 3) | (CFU/g) | (n = 3) | (CFU/g) |

25° C. - 12% DBM

| | | | | | | | | | | |
|---|---|---|---|---|---|---|---|---|---|---|
| 0 | 208.33 | 3.67 | 180.33 | 3.60 | 196.67 | 3.64 | 200 | 3.65 | 199.67 | 3.65 |
| 1 | 158 | 3.55 | 5 | 2.05 | 85.23 | 3.28 | 140 | 3.49 | 145.33 | 3.51 |
| 2 | 105.33 | 3.37 | *ND | 0.00 | 12.8 | 2.45 | 80 | 3.25 | 110 | 3.39 |
| 3 | 80 | 3.25 | *ND | — | *ND | 0.00 | 54 | 3.08 | 90 | 3.30 |
| 4 | 55.67 | 3.09 | *ND | — | *ND | — | 32.33 | 2.86 | 60.67 | 3.13 |
| 5 | 45.33 | 3.00 | *ND | — | *ND | — | 23.67 | 2.72 | 57 | 3.10 |
| 6 | 32.67 | 2.86 | *ND | — | *ND | — | 16.67 | 2.57 | 38 | 2.93 |

5° C. - 12% DBM

| | | | | | | | | | | |
|---|---|---|---|---|---|---|---|---|---|---|
| 0 | 208.33 | 3.67 | 180.33 | 3.60 | 196.72 | 3.64 | 200 | 3.65 | 199.76 | 3.65 |
| 1 | 188.67 | 3.62 | 10 | 2.35 | 85.23 | 3.28 | 140 | 3.49 | 165 | 3.56 |
| 2 | 146 | 3.51 | *ND | — | 12.8 | 2.45 | 80 | 3.25 | 131.3 | 3.47 |
| 3 | 105.67 | 3.37 | *ND | — | 3 | 1.82 | 71 | 3.20 | 90.67 | 3.30 |
| 4 | 88 | 3.29 | *ND | — | *ND | — | 53.42 | 3.07 | 61 | 3.13 |
| 5 | 62.67 | 3.14 | *ND | — | *ND | — | 32.78 | 2.86 | 57 | 3.10 |
| 6 | 44 | 2.99 | *ND | — | *ND | — | 18.65 | 2.62 | 38 | 2.93 |

32° C. - 12% DBM

| | | | | | | | | | | |
|---|---|---|---|---|---|---|---|---|---|---|
| 0 | 208.33 | 3.67 | 180.33 | 3.60 | 196.67 | 3.64 | 200 | 3.65 | 199.67 | 3.65 |
| 1 | 168.67 | 3.57 | 9 | 2.30 | 85.33 | 3.28 | 143.67 | 3.50 | 175 | 3.59 |
| 2 | 136 | 3.48 | *ND | — | 32.67 | 2.86 | 115.33 | 3.41 | 130 | 3.46 |
| 3 | 110.33 | 3.39 | *ND | — | 11 | 2.39 | 89 | 3.30 | 100.33 | 3.35 |
| 4 | 92 | 3.31 | *ND | — | *ND | — | 52.33 | 3.07 | 90.67 | 3.30 |
| 5 | 75.33 | 3.22 | *ND | — | *ND | — | 29.67 | 2.82 | 77 | 3.23 |
| 6 | 53.67 | 3.08 | *ND | — | *ND | — | 19.67 | 2.64 | 58 | 3.11 |

### SS Agar and MRS Agar Plates After Different Treatments (FIGS. 4, 5, and 6)

FIG. 4 shows the results obtained after inoculating 100 μL of the product obtained at the end of the protocol shown in FIG. 2, on MRS agar plates. The colonies belonged to the genus Lactobacillus and the genus Propionibacterium.

FIG. 5 shows the result of the plates obtained after performing the protocol in soybean meal. a. Comparison of Lactic-Propionic mix with control condition at 24 h post-contamination. b: Comparison of Lactic-Propionic mix at 48 h post-contamination.

FIG. 6 shows a comparison of Salmonella concentration after 24 hours from protocol development (FIG. 2) with different mixes (Lactic-Propionic and Lactic mixes)

### Moisture and Effectiveness of Soybean Meal Mix (FIG. 8)

After oil is stripped from the soybean flakes during the extraction process, the latter goes through a desolventizer to evaporate the residual hexane remaining after extraction. This process adds moisture to the flakes, since desolventizing is a process that uses steam. Once the soybean flake is desolventized, it is dried to obtain the desired moisture level. The moisture content assessed on a dry basis of a typical meal product is around 11-12% DBM; however, at present we can find meals on the market with a moisture content ranging from 8-13% DBM. The effectiveness of the mix of the invention was tested in meals with different moisture contents. The results showed that in matrixes with a “typical” water content (11-12%) the Lactic-Propionic mix was better than the Lactic mix, but that the difference was even greater when water availability was as low as 8% DBM (FIG. 8). Table 3 below shows the results illustrated in FIG. 8.

TABLE 3
25° C. - 8% DBM

| | | | MW Lactic- | | | | MW P. | | MW Propionic | |
|---|---|---|---|---|---|---|---|---|---|---|
| | Meal Control | | Propionic Mix | | MW Lactic Mix | | shermanii | | Acid | |
| Time | Average | LOG10 | Average | LOG10 | Average | LOG10 | Average | LOG10 | Average | LOG10 |
| (hours) | (n = 3) | (CFU/g) | (n = 3) | (CFU/g) | (n = 3) | (CFU/g) | (n = 3) | (CFU/g) | (n = 3) | (CFU/g) |
| 0 | 208.33 | 3.67 | 180.32 | 3.602831 | 196.67 | 3.64 | 200 | 3.65 | 199.67 | 3.65 |
| 12 | 168.67 | 3.57 | 53 | 3.071063 | 85.33 | 3.28 | 143.67 | 3.50 | 175 | 3.59 |
| 24 | 136 | 3.48 | 13 | 2.460731 | 32.67 | 2.86 | 115.33 | 3.41 | 130 | 3.46 |
| 36 | 110.67 | 3.39 | *ND | — | 16 | 2.55 | 89 | 3.30 | 100.33 | 3.35 |
| 48 | 92 | 3.31 | *ND | — | 2 | 1.65 | 52.33 | 3.07 | 90.67 | 3.30 |
| 60 | 75.33 | 3.22 | *ND | — | 1 | 1.35 | 29.67 | 2.82 | 77 | 3.23 |
| 72 | 53.67 | 3.08 | *ND | — | *ND | — | 19.67 | 2.64 | 58 | 3.11 |
| 84 | 29.67 | 2.82 | *ND | — | *ND | — | 4 | 1.95 | 32.33 | 2.85 |
| 96 | 19 | 2.63 | *ND | — | *ND | — | 1 | 1.35 | 15 | 2.52 |

| DBM | a_{w} |
|---|---|
| 13.75 | 0.7 |
| 11.24 | 0.635 |
| 10.27 | 0.584 |
| 8.83 | 0.557 |
| 6.41 | 0.406 |
| 5.48 | 0.324 |
| 4.2 | 0.232 |
| 3.64 | 0.182 |
| 2.82 | 0.14 |
| 1.48 | 0.06 |

### EXAMPLE 2

### Protection Against Fungi and Yeasts (FIG. 9)

A common problem of grain and soybean meal processing is the occurrence of mycotoxins. These problematic metabolites are often synthesized by fungi of the genera Aspergillus, Penicillium and Fusarium. The detection of mycotoxins in meal products means that a fungus is or has been present in the matrix.

Due to this problem, it was decided to assess the antifungal power of the Lactic-Propionic mix, and to compare it with the Lactic mix. The antifungal properties of propionic acid are well known, and so is the antifungal activity of bacteria of the order Actinomycetales, such as P. shermanii.

The protocol used for this purpose shared many similarities with the protocol used to assess the effectiveness against Salmonella, except that in this case, the meal product was infected with 10⁶ conidia of Aspergillus niger ATCC 16404. For the determination of fungal CFU, 20 grams of soybean meal were added to 180 mL of sterile tap water with Tween 80, which was vigorously stirred. Serial dilutions of the sample were carried out in order to perform recounting on the appropriate plates, in a Czapek-Dox agar medium. Table 4 below shows the results illustrated in FIG. 9.

TABLE 4
25° C. - 12% DBM

| | | | MW Lactic- | | | | MW P. | | MW Propionic | |
|---|---|---|---|---|---|---|---|---|---|---|
| | Meal Control | | Propionic Mix | | MW Lactic Mix | | shermanii | | Acid | |
| Time | Average | LOG10 | Average | LOG10 | Average | LOG10 | Average | LOG10 | Average | LOG10 |
| (days) | (n = 3) | (CFU/g) | (n = 3) | (CFU/g) | (n = 3) | (CFU/g) | (n = 3) | (CFU/g) | (n = 3) | (CFU/g) |
| 0 | 198.33 | 3.64 | 189.33 | 3.62 | 196.67 | 3.64 | 200 | 3.65 | 199.76 | 3.65 |
| 1 | 188.67 | 3.62 | 105 | 3.37 | 185.33 | 3.61 | 140 | 3.49 | 152 | 3.53 |
| 2 | 176 | 3.59 | 33 | 2.87 | 152.67 | 3.53 | 80 | 3.25 | 121.33 | 3.43 |
| 3 | 170.33 | 3.58 | 2 | 1.65 | 123.33 | 3.44 | 54 | 3.08 | 91.67 | 3.31 |
| 4 | 166 | 3.57 | *ND | — | 96.67 | 3.33 | 32.33 | 2.86 | 78 | 3.24 |
| 5 | 159.33 | 3.55 | *ND | — | 45 | 3.00 | 23.67 | 2.72 | 57 | 3.10 |
| 6 | 155.67 | 3.54 | *ND | — | 22 | 2.69 | 16.67 | 2.57 | 38 | 2.93 |

### EXAMPLE 3

### Contamination and Recontamination After Treatment With the Lactic-Propionic Mix (FIG. 10)

Transportation of meal products is a very complex task, often associated with long periods of time (up to one month of logistics). During all this time, re-contamination is very likely to occur. Even if the meal is not exposed to contact with undesirable microorganisms during its transportation, it may still be contaminated when arriving at the port of destination. For this reason it was decided to study the response of meals protected with different solutions, by contaminating them at different times after protection. Given the complexity of the logistics of meal products, they were tested at two different times: initial contamination and recontamination on the first week after treatment; and, contamination on the fourth week with subsequent recontamination on the fifth week after treatment. In all cases, itl was contaminated and recontaminated with Salmonella solutions whose concentration was approximately 10⁶ CFU/mL (as previously described). Table 5 below shows the results illustrated in FIG. 10.

TABLE 5

| | | | MW Lactic- | | | | MW P. | | MW Propionic | |
|---|---|---|---|---|---|---|---|---|---|---|
| | Meal Control | | Propionic Mix | | MW Lactic Mix | | shermanii | | Acid | |
| Time | Average | LOG10 | Average | LOG10 | Average | LOG10 | Average | LOG10 | Average | LOG10 |
| (days) | (n = 3) | (CFU/g) | (n = 3) | (CFU/g) | (n = 3) | (CFU/g) | (n = 3) | (CFU/g) | (n = 3) | (CFU/g) |

25° C. - 12% DBM

| | | | | | | | | | | |
|---|---|---|---|---|---|---|---|---|---|---|
| 0 | 208.3 | 3.67 | 180.33 | 3.60 | 196.72 | 3.64 | 200 | 3.65 | 199.76 | 3.65 |
| 1 | 168.7 | 3.57 | 53 | 3.07 | 85.23 | 3.28 | 143.6 | 3.50 | 175 | 3.59 |
| 2 | 136 | 3.48 | 13 | 2.46 | 32.8 | 2.86 | 115.3 | 3.41 | 130 | 3.46 |
| 3 | 110.3 | 3.39 | *ND | — | 16 | 2.55 | 89 | 3.30 | 100.3 | 3.35 |
| 4 | 92 | 3.31 | *ND | — | 2 | 1.65 | 52.43 | 3.07 | 90.5 | 3.30 |
| 5 | 75.33 | 3.22 | *ND | — | 1 | 1.35 | 29.87 | 2.82 | 77 | 3.23 |
| 6 | 53.67 | 3.08 | *ND | — | *ND | — | 19.85 | 2.64 | 58 | 3.11 |
| 7 | 29.67 | 2.82 | *ND | — | *ND | — | 4 | 1.95 | 32 | 2.85 |

RECONTAMINATION

| | | | | | | | | | | |
|---|---|---|---|---|---|---|---|---|---|---|
| 8 | 219 | 3.69 | 220 | 3.69 | 205 | 3.66 | 216 | 3.68 | 215 | 3.68 |
| 9 | 198 | 3.64 | 43.67 | 2.99 | 97.67 | 3.34 | 164.67 | 3.56 | 189.33 | 3.62 |
| 10 | 176.7 | 3.59 | 6 | 2.12 | 42.67 | 2.98 | 123.33 | 3.44 | 144.33 | 3.51 |
| 11 | 148.3 | 3.52 | 3 | 1.82 | 11.33 | 2.40 | 99 | 3.34 | 109 | 3.38 |
| 12 | 115.7 | 3.41 | *ND | — | 4 | 1.95 | 78.33 | 3.24 | 96.67 | 3.33 |
| 13 | 98.67 | 3.34 | *ND | — | 1 | 1.35 | 60.67 | 3.13 | 70.67 | 3.20 |
| 14 | 85.33 | 3.28 | *ND | — | *ND | — | 52.67 | 3.07 | 48.67 | 3.03 |
| 15 | 60.33 | 3.13 | *ND | — | *ND | — | 39.67 | 2.95 | 40 | 2.95 |

25° C. - 12% DBM

| | | | | | | | | | | |
|---|---|---|---|---|---|---|---|---|---|---|
| 30 | 208.3 | 3.67 | 194.33 | 3.64 | 216.67 | 3.68 | 208 | 3.66 | 199.76 | 3.65 |
| 31 | 172.7 | 3.58 | 73.67 | 3.21 | 97.33 | 3.33 | 134.67 | 3.48 | 175 | 3.59 |
| 32 | 146 | 3.51 | 25 | 2.74 | 52.67 | 3.07 | 125.33 | 3.44 | 130 | 3.46 |
| 33 | 123.7 | 3.44 | 3 | 1.82 | 16 | 2.55 | 100 | 3.35 | 120.3 | 3.43 |
| 34 | 96.33 | 3.33 | *ND | — | 9 | 2.30 | 82.33 | 3.26 | 90.5 | 3.30 |
| 35 | 72.33 | 3.21 | *ND | — | 2 | 1.65 | 59.67 | 3.12 | 77 | 3.23 |
| 36 | 57.33 | 3.11 | *ND | — | *ND | — | 29.33 | 2.81 | 58 | 3.11 |
| 37 | 49.67 | 3.04 | *ND | — | *ND | — | 14.67 | 2.51 | 39 | 2.94 |

RECONTAMINATION

| | | | | | | | | | | |
|---|---|---|---|---|---|---|---|---|---|---|
| 38 | 250 | 3.74 | 202 | 3.65 | 205 | 3.66 | 216 | 3.68 | 247 | 3.74 |
| 39 | 193 | 3.63 | 63.67 | 3.15 | 97.67 | 3.34 | 154.67 | 3.54 | 189 | 3.62 |
| 40 | 183.7 | 3.61 | 26.67 | 2.77 | 62.33 | 3.14 | 133.33 | 3.47 | 174.34 | 3.59 |
| 41 | 162.7 | 3.56 | 5 | 2.05 | 37.67 | 2.92 | 92.67 | 3.31 | 156.34 | 3.54 |
| 42 | 135.7 | 3.48 | *ND | — | 14.33 | 2.50 | 78.33 | 3.24 | 136.34 | 3.48 |
| 43 | 119.7 | 3.43 | *ND | — | 8 | 2.25 | 60.67 | 3.13 | 111.67 | 3.39 |
| 44 | 96.33 | 3.33 | *ND | — | 2.33 | 1.72 | 42.67 | 2.98 | 88.34 | 3.29 |
| 45 | 80.33 | 3.25 | *ND | — | 1.33 | 1.47 | 19 | 2.63 | 77 | 3.23 |

In all cases the Lactic-Propionic mix gave the best results.

### EXAMPLE 4

### Method of Application (FIG. 11)

Meal products are a solid, anhydrous and heterogeneous matrix. Mixing the ferment produced by different mixes within such matrix not simple, particularly taking into account that moisture cannot exceed a certain value. A compromise solution between the percent of matrix protein, moisture and other parameters should be reached. In order to properly distribute the ferment, it was decided to use a combination of devices. In the fermentation plant a tank capable of holding for a few hours the fermented mix was added, while in the meal production plant a sprinkler head, and a screw mixer were added. Thus, fermentation of all strains was started so that all processes would be completed at the same time, and in equal volumes. After completion of fermentation the ferments were sent to a buffer tank refrigerated at 4° C. in batches that would be consumed every 24 hours, in this way any potential antagonistic effect between different ferments was avoided. The Propionic-Lactic mix was mixed with a saline solution to increase dispensed volumes, thus supplying a homogeneous ferment mix on each meal particle. Dispensed volumes will heavily depend on the concentrations obtained from fermentation, the desired level of protection, and the intended added cost to meal production.

The meal was fed by gravity onto a screw conveyor, passed through an area where there was a “cloud of ferment” sprayed through a metered nozzle, and then this “wet” meal entered into a screw mixer.

This method provides a protected meal product using the mix of the invention. The method further provides fine-adjustment capabilities to moisture variations as small as 0.2% of the moisture content of the meal product.

1. A synergistic composition comprising a mix of bacteria of the genera Lactobacillus and Propionibacterium particularly useful to reduce or eliminate contamination by bacteria of the genus Salmonella and by mycotoxin-producing fungi in soybean meal and its derivatives comprising:
Lactobacillus casei ATCC 393,
Lactobacillus fermentum ATCC 9338,
Lactobacillus gasseri ATCC 33323,
Lactobacillus plantarum ATCC 14917,
Lactobacillus rhamnosus ATCC 7469, and
Propionibacterium freudenreichii subsp. shermanii ATCC 9614.

2. The synergistic composition of claim 1, characterized in that said mix of bacteria has a cell concentration in the range of 10⁵-10¹¹ CFU/mL.

3. The synergistic composition of claim 1 characterized in that all said bacteria are included in equal quantities and have similar CFU/mL concentrations.

4. The synergistic composition of claim 2 characterized in that in said mix of strains having a cell concentration in the range of 10⁵-10¹¹ CFU/mL, the most concentrated strain is not more than 1000 times more concentrated (in CFU/mL) than the least fermented strain.

5. (canceled)

6. (canceled)

7. The synergistic composition of claim 1 effective for both initial contamination and recontamination during the first week after treatment; and contamination during the fourth week with subsequent recontamination on the fifth week after treatment.

8. (canceled)

9. The synergistic composition of claim 1, in which said mycotoxin-producing fungi are conidia of Aspergillus niger.

10. The synergistic composition of claim 1, characterized in that the bacteria are suspended in a Modified MRS broth for fermentation comprising: (NH₄)NO₃ 1 g/L, Yeast Extract 20 g/L, Glucose 30 g/L, Sorbitan Monoleate 1 mL/L, K₂HPO₄ 2 g/L, Sodium Acetate 5 g/L, MgSO₄ 0.2 g/L, MnSO₄ 0.05 g/L.

11. (canceled)

12. Method of application of the synergistic composition of claim 1 characterized in that the meal is caused to fall by gravity into a screw conveyor, to pass through an area where there is a cloud of such synergistic composition sprayed by a metered nozzle, and then this wet meal is conveyed into a mixing screw.


SEQUENCE LISTING (12 sequences)

SEQ ID NO: 1 - DNA, 24 bp, Artificial Sequence
  Forward primer for 16S DNA region of L. casei
cgagttttggtcgatgaacggtgc

SEQ ID NO: 2 - DNA, 21 bp, Artificial Sequence
  Reverse primer for 16S DNA region of L. casei
atcatcgccttggtgagccgt

SEQ ID NO: 3 - DNA, 20 bp, Artificial Sequence
  Forward primer for 16S DNA region of L. fermentum
caacgagtggcggacgggtg

SEQ ID NO: 4 - DNA, 20 bp, Artificial Sequence
  Reverse primer for 16S DNA region of L. fermentum
tgcaccgcaggtccatccag

SEQ ID NO: 5 - DNA, 20 bp, Artificial Sequence
  Forward primer of 16S DNA region of L. gasseri
ggcggctctctggtctgcaa

SEQ ID NO: 6 - DNA, 20 bp, Artificial Sequence
  Reverse primer for 16S DNA region of L. gasseri
cgggcccccgtcaattcctt

SEQ ID NO: 7 - DNA, 20 bp, Artificial Sequence
  Forward primer of 16S DNA region of L. plantarum
ggaccgcatggtccgagctt

SEQ ID NO: 8 - DNA, 20 bp, Artificial Sequence
  Reverse primer of 16S DNA region of L. plantarum
cgggcccccgtcaattcctt

SEQ ID NO: 9 - DNA, 20 bp, Artificial Sequence
  Forward primer of 16S DNA region of L. rhamnosus
tggacccgcggcgtattagc

SEQ ID NO: 10 - DNA, 22 bp, Artificial Sequence
  Reverse primer of 16S DNA region of L. rahmnosus
tgcctacgtattaccggctggc

SEQ ID NO: 11 - DNA, 20 bp, Artificial Sequence
  Forward primer of 16S-23S DNA region of L. shermanii
tgaccgtagattgtcggctg

SEQ ID NO: 12 - DNA, 20 bp, Artificial Sequence
  Reverse primer of 16S-23S DNA region of L. shermanii
caaacacggggaacaaccac